(19) United States
(12) Patent Application Publication
(10) Pub. No.: US 2013/0210752 A1
(43) Pub. Date: Aug. 15, 2013

(54) USING A NOVEL HIGH-THROUGHPUT YEAST CELL-BASED SCREENING PLATFORM TO IDENTIFY HMG-COA REDUCTASE INHIBITORS FROM NATURAL PRODUCTS AND THE USES OF SUCH INHIBITORS THEREOF

(71) Applicant: Hong Kong Baptist University, (US)

(72) Inventors: Wendy Wen Luan HSIAO, Hong Kong (HK); William Chi-Shing TAI, Hong Kong (HK); Zhi-Hong JIANG, Hong Kong (HK)

(73) Assignee: HONG KONG BAPTIST UNIVERSITY, HK (HK)

(21) Appl. No.: 13/762,400

(22) Filed: Feb. 8, 2013

Related U.S. Application Data
(60) Provisional application No. 61/597,162, filed on Feb. 9, 2012.

(51) Int. Cl. C12Q 1/26

(52) U.S. Cl. CPC C12Q 1/26
USPC 514/26; 435/25; 536/5

(57) ABSTRACT

Methods of screening, identifying and isolating HMG-CoAR inhibitors are disclosed herein. In particular, said method is useful for screening natural raw products. The present invention also provides potent HMG-CoAR inhibitors for treatment and prevention of diseases and conditions associated with elevated cholesterol and/or blood lipid levels.

### CROSS-REFERENCE TO RELATED APPLICATIONS

The present application claims priority of U.S. provisional application No. 61/597,162 filed Feb. 9, 2012, and which the disclosure is hereby incorporated by reference.

### FIELD OF INVENTION

The present invention relates to a method to identify inhibitors for the enzyme 3-hydroxy-3-methylglutaryl coenzyme A reductase (HMG-CoAR). In particular, the present invention relates to method for identifying and isolating HMG-CoAR inhibitors from natural products. The present invention also provides novel HMG-CoAR inhibitors that are useful in the treatment and/or prevention of diseases and symptoms associated with increased blood lipid and cholesterol level.

### BACKGROUND OF INVENTION

The enzyme 3-hydroxy-3-methylglutaryl coenzyme A reductase (HMG-CoAR) is the key rate limiting enzyme of the mevalonate pathway, which is a highly conserved pathway in all eukaryotic cells for cholesterol production. Therefore, compounds having HMG-CoAR inhibititory activity are currently used in lowering blood cholesterol level, and thereby treating and/or preventing diseases and symptoms associated with increased cholesterol level.

HMG-CoAR inhibitors like other drugs, are associated with various adverse side effects. Among these adverse effects, the most common ones are raised liver enzyme levels and muscle problems. While current HMG-CoAR inhibitors are well tolerated by most patients, the adverse side effects associated therewith can potentially generate a significant public health impact as these drugs are prescribed on wide scale.

U52009/0285915 A1 and PCT/182009/005461 that disclosed synergistic blood lipid lowering composition comprising synergistically effect amounts of KDC (Ku Ding Cha), HL (Huanglian) and at least one of the herbal extracts including Gynostemma pentaphyllum (Gypenosides) in combination; to the best of our knowledge, the present invention is previously unknown and not anticipated, wherein the total saponin extract of Gynostemma pentaphyllum (GpS) on its own can inhibit the HMG-CoAR activity.

The objective of the present invention is to provide an efficient, high-throughput and reproducible method for screening HMG-CoAR inhibitors for the treatment of blood cholesterol and blood lipid related disorders and/or conditions. In particular, the present invention provides a method for identifying and isolating novel HMG-CoAR inhibitors from natural products.

Citation or identification of any reference in this section or any other section of this application shall not be construed as an admission that such reference is available as prior art for the present application.

### SUMMARY OF INVENTION

In one aspect, the present invention provides a novel screening method using yeast cells to identify HMG-CoAR inhibitors. The HMG-CoAR inhibitor screening method provided by the present invention is a reproducible, efficient, low-cost and highly accurate as compared to conventional screening methods using mammalian cell lines. Another aspect of the invention provides a kit for identifying HMG-CoAR inhibitors. In another aspect, the present invention provides a yeast cell-based activity-guided fractionation assay screening platform to identify and isolate HMG-CoAR inhibitors from natural products.

In another aspect, the present invention further provides composition for treating and preventing diseases and conditions associated with elevated cholesterol and blood lipid level, such as hypercholesterolemia and hyperlipidemic. The present composition for treating and preventing diseases and conditions associated with elevated cholesterol and blood lipid level comprises an effective amount of a extract of Gynostemma pentaphyllum (Gp), compounds isolated from Gp, total saponins from Gp, fraction from total saponins from Gp, isolated saponins from Gp, a compound having Formula (I) or Formula (II) or a combination thereof. Formula (I) and Formular (II) are

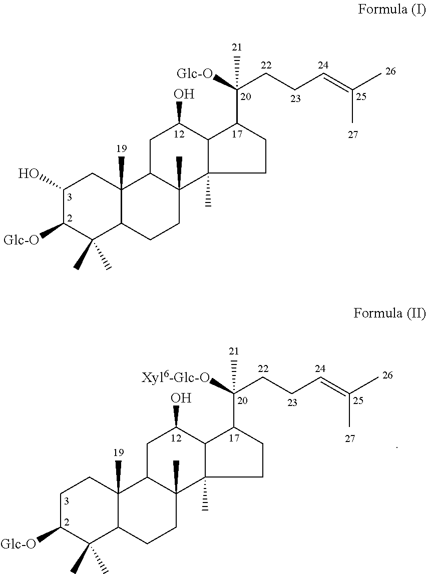

The present composition can also be administered in conjunction with other compounds effective in treating diseases and/or condition associated with elevated cholesterol and/or blood lipid level.

Those skilled in the art will appreciate that the invention described herein is susceptible to variations and modifications other than those specifically described.

The invention includes all such variations and modifications. The invention also includes all of the steps and features referred to or indicated in the specification, individually or collectively and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 demonstrates the growth inhibitory effect of simvastatin, a cholesterol lowering drug, in yeast cells with (left) or without (right) the presence of 10 mg/ml mevalonate (Mev). *p<0.05, **p<0.01(compare with control without Mev); ^{##}p<0.01 (compare with its corresponding dosage without Mev).

FIG. 2 shows the growth inhibitory effect of GpS in yeast cells with (left) or without (right) the presence of 10 mg/ml mevalonate. *p<0.05, ***p<0.001(compare with Ctl without Mev); ^{#}p<0.05, ^{###}p<0.001 (compare with its corresponding dosage without Mev).

FIG. 3 shows the growth inhibitory effect of isolated fractions (500 μg/ml fractions 7-10) from GpS with (left) or without (right) mevalonate in yeast cells. ***p<0.001 (compare with Ctl without Mev); ^{###}p<0.001 (compare with Fr.7 without Mev).

FIGS. 4 depict the total ion chromatograms (TIC) of Fraction 7 (FIG. 4A) of gypenosides and the obtained six compounds (FIG. 4B-4G).

FIG. 5 depicts the growth inhibitory effects of compounds (300 μg/ml 7B1, 300 μg/ml 7B5, 300 μg/ml 7C5, 300 μg/ml 7D7, 150 μg/ml 7E1 and 300 μg/ml 7E2) isolated from Fraction 7 in yeast cells with (left) or without (right) mevalonate. ** p<0.01 (compare with Ctl without Mev); ^{##}p<0.01, ^{###}p<0.001 (compare with 7E1 and 7E2 without Mev).

FIG. 6 shows the HMG-CoA activity of Pravastatin, 60 μg/ml7E1 and 60 μg/ml 7E2. * p<0.05, *** p<0.001. Commercial HMG-CoAR activity assay kit (Sigma, USA) is used in this experiment. The HMG-CoAR activity assay is based on the spectrophotometric measurement of the decrease in absorbance at 340 nm, which represents the oxidation of NADPH by the catalytic subunit of HMGR in the presence of the substrate of HMG-CoA. Pravastatin (Positive control), 7E1 or 7E2 are tested in this assay.

### DETAILED DESCRIPTION OF INVENTION

The present invention is not to be limited in scope by any of the specific embodiments described herein. The following embodiments are presented for exemplification only.

Conventional HMG-CoAR inhibitors screening methods rely on mammalian cell line is both expensive and time consuming. The present invention provides a novel screening method to identify potential HMG-CoAR inhibitors that is low-cost, easily accessible, reproducible and efficient.

Previous studies demonstrated the structural and functional conservation of HMG-CoAR in yeast (Saccharomyces cerevisiae) and humans. Due to the functional conservation of the HMG-CoAR between yeast and humans, yeast is used in the present invention to identify the potential HMG-CoAR inhibitors for treatment and/or prevention of diseases associated with increased cholesterol level, e.g. hypercholesterolemia.

In yeast cells, there are two genes encoding HMG-CoAR, designated HMG1 and HMG2. Deletion of either one of the genes will only have a subtle growth defect but deletion of both of the genes will result in a lethal phenotype in yeast cells. HMG1 contributes more than 70% of HMG-CoAR activity in yeast cells. Expression of HMG1 in yeast cells that lack both HMG1 and HMG2 genes can restore the viability.

The present invention provides a screening method to identify potential HMG-CoAR inhibitors. The present method comprises providing a yeast strain which lacks HMG1 and HMG2 genes but containing a HMG1 overexpression plasmid, contacting said yeast strain with a test material. If the test material can inhibit the HMG1 activity or expression, the yeast cells will die. In order to validate the death of yeast cells is due to inhibition of HMG1 by the test material, the present invention also includes adding mevalonate compound, which is a downstream product of the HMG-CoAR. HMG-CoAR inhibitory activity of the test material is confirmed if the cell viability of the yeast strain is unaffected by the test material in the presence of mevalonate.

In one embodiment, the test material and mevalonate can be added to the yeast strain at the same time. In yet another embodiment, the present method is performed under room-temperature.

In another embodiment, the test material is a natural product. The term “natural product” used herein referred to chemical compound or substance produced by or extracted from a living organism or the living organism itself. Examples of living organism include, but are not limited to, plants, vertebrates, non-vertebrates or microorganisms. The present invention further comprises separating the natural product, which has been confirmed to possess HMG-CoAR inhibition activity into at least two fractions and confirming the HMG-CoAR inhibition activity of the fractions. In another embodiment, the present invention purifies compounds from natural product fractions, which are confirmed to have HMG-CoAR inhibitor activity and confirming the HMG-CoAR inhibition activity of the purified compounds. In yet another embodiment, HMG-CoAR inhibitor identified and isolated by the present screening method is useful in treating and preventing diseases and conditions associated with elevated cholesterol and blood lipid levels.

In one embodiment, the invention provides a kit for identifying potential HMG-CoAR inhibitors. The kit comprises a yeast strain which lacks HMG1 and HMG2 genes but containing a HMG1 overexpression plasmid; melovanote for verification of HMG-CoAR inhibitory activity and cell viability testing means.

Inhibition of an NADPH dependent HMG-CoAR that catalyzes the reduction of HMG-CoA to mevalonate and provides feedback regulation in the cholesterol biosynthetic pathway is the main strategy for lowering blood cholesterol levels. Since 1970s, statins or HMG-CoAR competitive inhibitors have been developed. Until now, they remain the drug of choice for lowering blood cholesterol in people with or at risk of cardiovascular disease (CVD). In widespread clinical use, such as those reported in La Rosa J C, He H, Vupputuri S. (1999) Effects of statins on risk of coronary disease. JAMA 282:2340-2346, statins have proven safe and effective for lowering LDL-C and providing primary and secondary prevention of CVD and coronary events, respectively. Beyond their lipid lowering effects, statins or HMG-CoAR inhibitors have also been reported in literatures such as Zhou Q, Liao J K. (2010) Pleiotropic effects of statins. Circ J 74:818-826, Zhou Q, Liao JK. (2009) Statins and cardiovascular disease: from cholesterol lowering to pleiotropy. Curr Pharm Des 15:467-478 and Athyros V G, Kakafika Al, Tziomalos K, Karagiannis A, Mikhailidis DP. (2009) Pleotropic effects of statins—clinical evidence. Curr Phar Des 15:479-489, to exhibit various pleiotropic effects such as reducing vascular inflammation, improving endothelial functions and normalizing the coagulation-fibrinolysis system that could be complementary in reducing CVD risk.

In another embodiment, the present invention provides composition for treating and preventing diseases and conditions associated with elevated cholesterol and blood lipid level, such as hypercholesterolemia and hyperlipidemic. The present composition for treating and preventing diseases and conditions associated with elevated cholesterol and blood lipid level comprises effective amount of a extract of Gynostemma pentaphyllum (Gp), compounds isolated from Gp, total saponins from Gp, fraction from total saponins from Gp, isolated saponins from Gp, a compound having Formula (I) or Formula (II) or a combination thereof.

Formula (I) and Formula (II) are

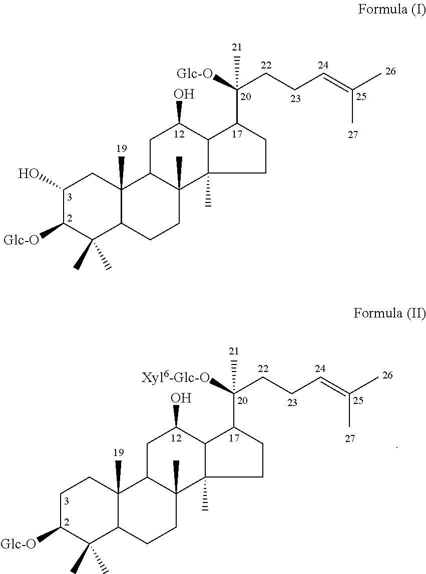

The compound having Formula (I) and (II) may be isolated from a natural product, such a herb. In another embodiment, the herb is Gynostemma pentaphyllum (Gp). The present composition can also be administered in conjunction with other compounds effective in treating diseases and/or condition associated with elevated cholesterol and/or blood lipid level.

### Working Examples

Yeast Strains and Growth Medium

Saccharomyces cerevisiae (MATa ade2-101 his3-200 hmg1::LYS2 hmg2:his3 lys2-80 met TRP1 ura3-52 with overexpression of yeast HMG1 containing plasmid) were used in the screening assay. Cells were grown at 30° C. in YEPD, consisting of 10 g/L yeast extract, 20 g/L Bacto-peptone and 20 g/L dextrose.

Yeast HMG-CoAR Screening Conditions

Yeast cells were grown overnight in YEPD medium to ^{˜}7.0 OD₆₆₀ at 30° C. Cells were then subculture to a fresh YEPD medium with starting ^{˜}0.1 starting OD₆₆₀ and grew for another 8 hours to ^{˜}1.2 OD₆₆₀. The assay was performed in a 96-well plate. 2.7×10⁴ cells, tested drugs/chemicals and/or mevalonate were added to each well in a final volume of 100 μl. All experiments were performed in triplicate. Cells were then grown for 16 hours at 30° C. and the inhibitory effect of HMG-CoAR determined by measuring the cell density OD₆₆₀ of each well by a micro-plate reader. Statin was used as a positive control and mevalonate supplement was used to validate the inhibitory effect of tested compounds on HMG-CoAR.

Determination of HMG-CoAR Activity in Vitro Assay

The HMG-CoAR activity was determined by the commercially available in vitro assay kit (Sigma, USA). The protocol of the assay was performed as described in the manufacture's manual. The HMG-CoAR activity assay is based on the spectrophotometric measurement of the decrease in absorbance at 340 nm, which represents the oxidation of NADPH by the catalytic subunit of HMGR in the presence of the substrate of HMG-CoA.

Gynostemma pentaphyllum Saponins (GpS)

GpS was purchased from Hui Zhou Shi Orient Plant Health Care SCL & Tech. CD., Ltd., HuiZhou of China with 85% purity.

### Example 1—Inhibitory Effect of Statin in Yeast Cells Growth

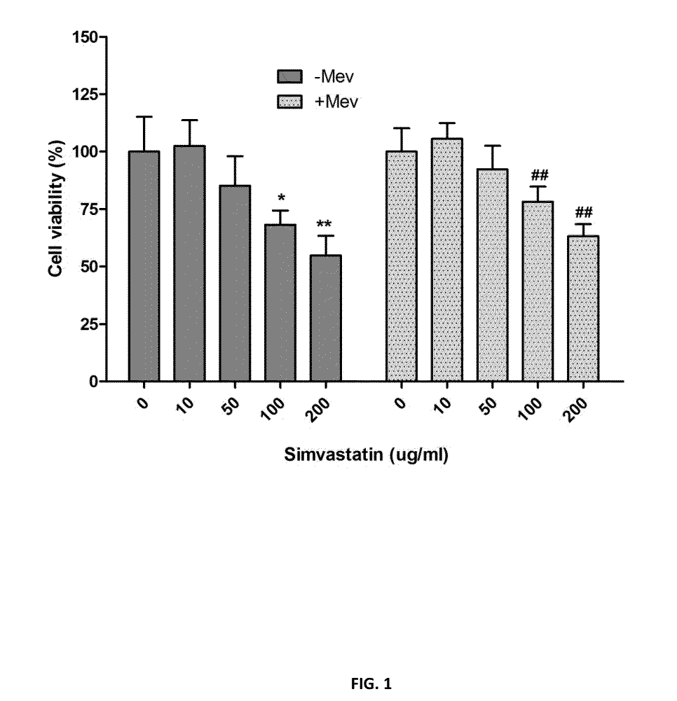

The growth of the yeast cells is first tested in the presence of known statin, simvastatin, which is widely administrated to hypercholesterolemia patients. Different concentrations of simvastatin are used in this study. Simvastatin is found to inhibit yeast cells growth in a dose-dependent manner (FIG. 1, left). The addition of mevalonate to the yeast cells reduces the inhibitory effect of simvastatin (FIG. 1, right). The results conclude that this yeast strain is suitable for identifying potential HMG-CoAR inhibitor(s) from herbal medicine and compounds. The results demonstrate that the present screening method can accurately identify HMG-CoAR inhibitors using yeast cell that overexpresses HMG1. Table 1 below compares the present screening method with conventional screening method using mammalian cells.

TABLE 1

| | | Conventional screening method using |
|---|---|---|
| | Present Invention | mammalian cell line |
| Preparative and | ~16 hours | ~48 hours |
| testing time | | |
| Culture medium | Low cost to maintain yeast | High cost to maintain mammalian cells |
| | cells (eg. Bacterial peptone, | (eg. Cell culture medium, Fetal Bovine |
| | Glucose, regular | Serum, growth factors, tissue culture |
| | plasticware) | coated plasticwares) |
| Facilities to | Room temperature | CO₂/37° C. humidified tissue culture |
| maintain cells | | incubator, Tissue culture hood. |
| Technical | Easy to handle | Well-trained personnel is required |
| requirements | | |
| Steps involved | Tested inhibitors and | Mammalian cells need to be culture on |
| | mevalonate can be added | the tissue culture coated plasticwares for |
| | to the yeast cells at the | 18-24 hours before adding the tested |
| | same time to test. | inhibitors and mevalonate to test. |

### Example 2—Mevalonate Rescues the Growth Inhibitory Effect GpS

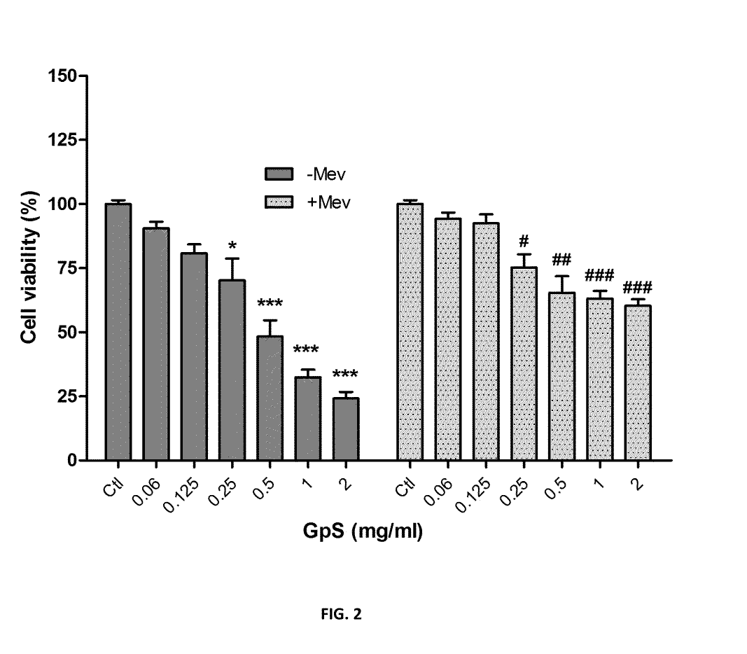

To isolate potential HMG-CoAR inhibitor(s) from GpS, growth inhibitory effect of GpS in yeast cells with or without the presence of mevalonate is tested. FIG. 2 shows that mevalonate rescues the growth inhibitory effect of the GpS in yeast cells. These results indicate that GpS contains HMG-CoAR inhibitor(s) that suppress the HMG-CoAR.

### Example 3—Fraction 7 isolated from GpS suppresses the growth of yeast cells

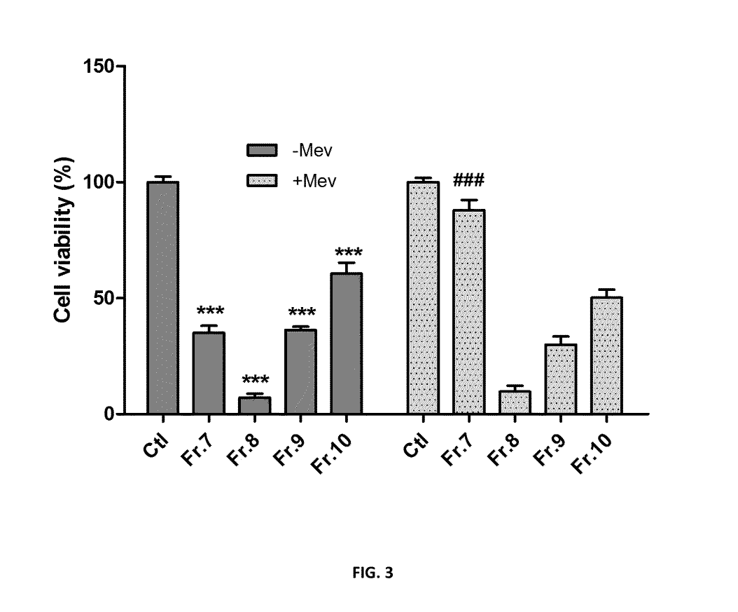

In order to isolate the HMG-CoAR inhibitor(s) in the GpS, the GpS is further separated into 10 different fractions and tested for growth inhibitory activity. Six fractions (Fraction 1-6) are found having no growth inhibitory effect on yeast cells (data not shown) which suggests these six fractions contain no HMG-CoAR inhibitor. Different degrees of inhibitory growth effects from Fractions 7-10 are observed in yeast cells (FIG. 3) but only the inhibitory effect from Fraction 7 is suppressed by mevalonate. The rest of the fractions (Fraction 8-10) only have mild to no restoration of cell growth by mevalonate (FIG. 3). The results conclude that fraction 7 contains HMG-CoAR inhibitor(s).

### Example 4—Gypenosides C7E1 and C7E2 isolated from Fraction 7 inhibit HMG-CoAR

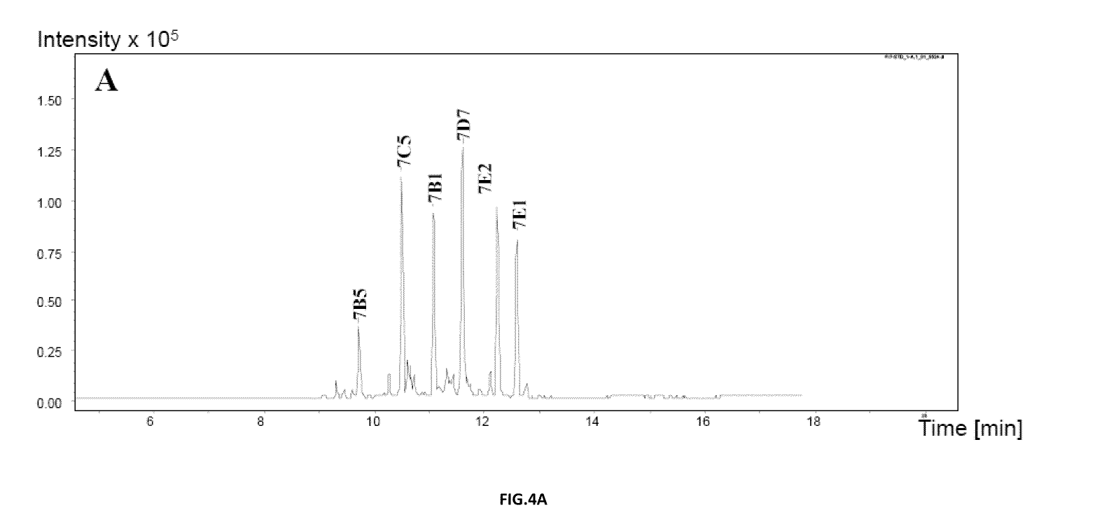

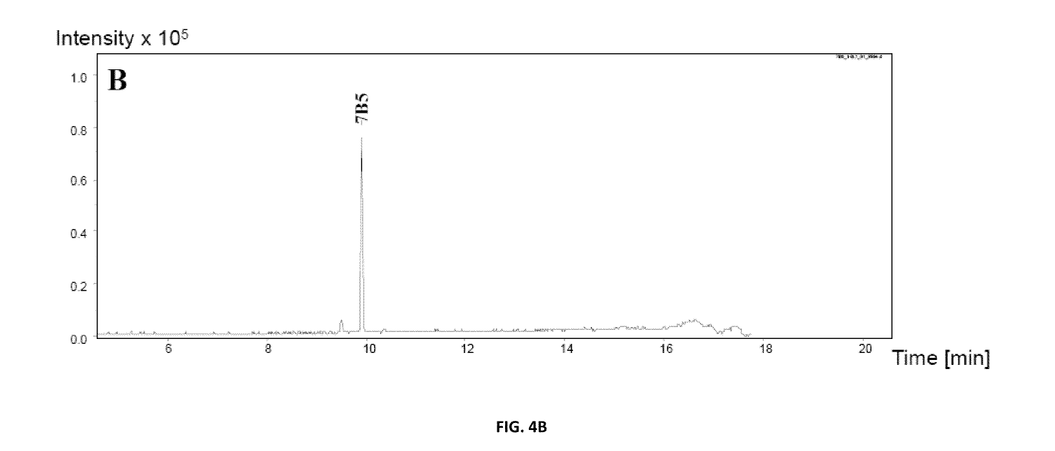

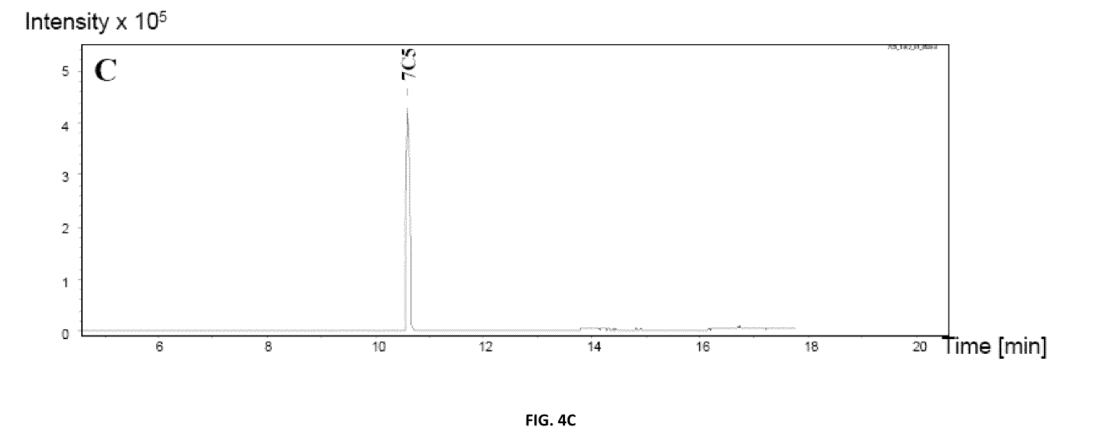

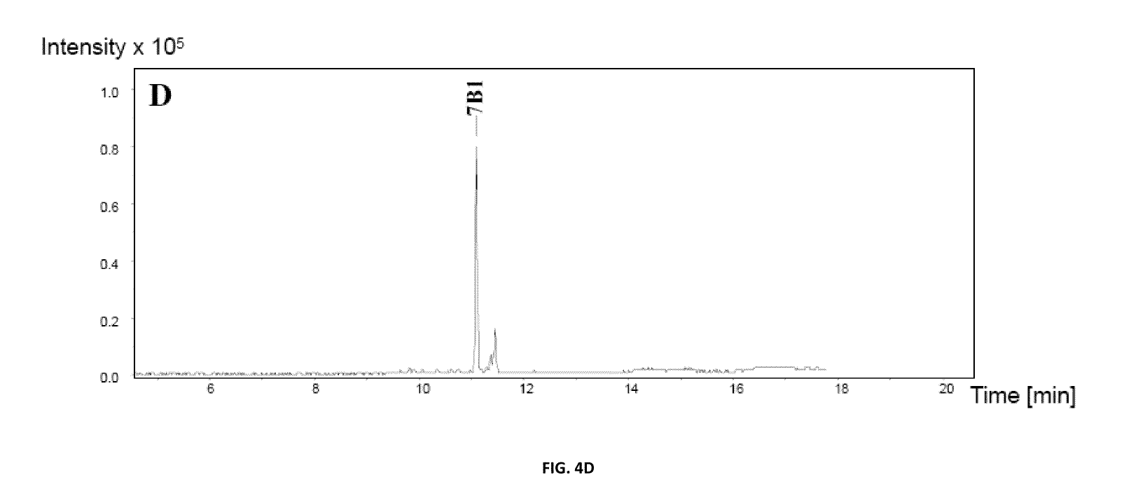

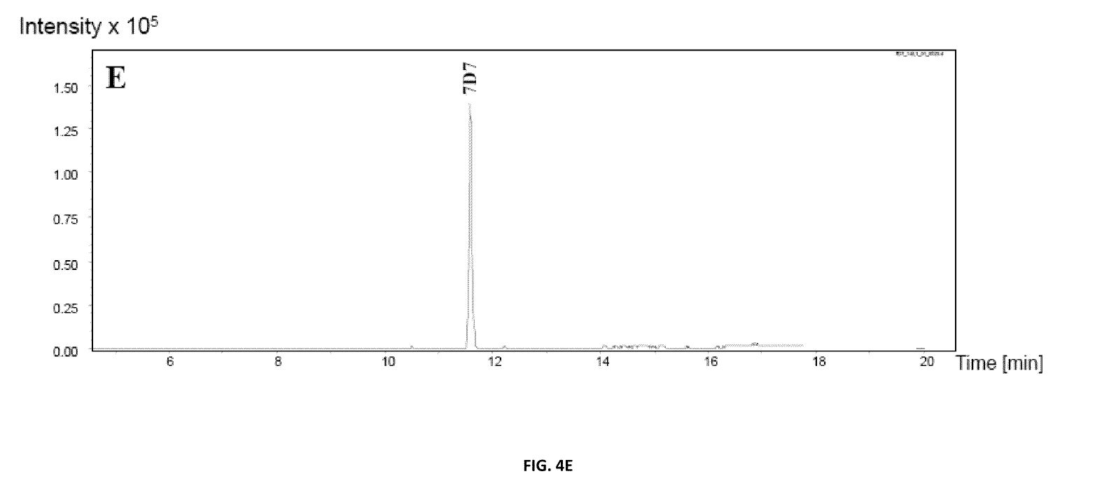

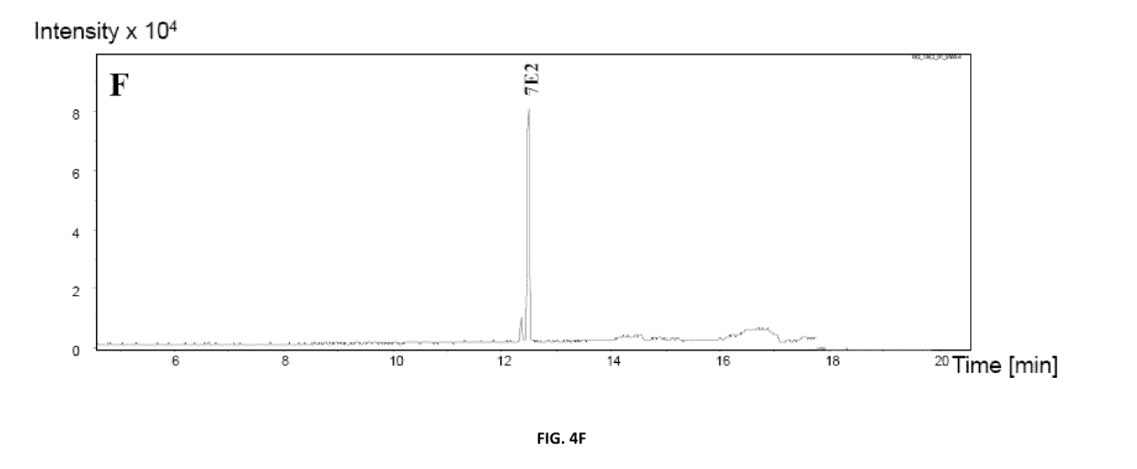

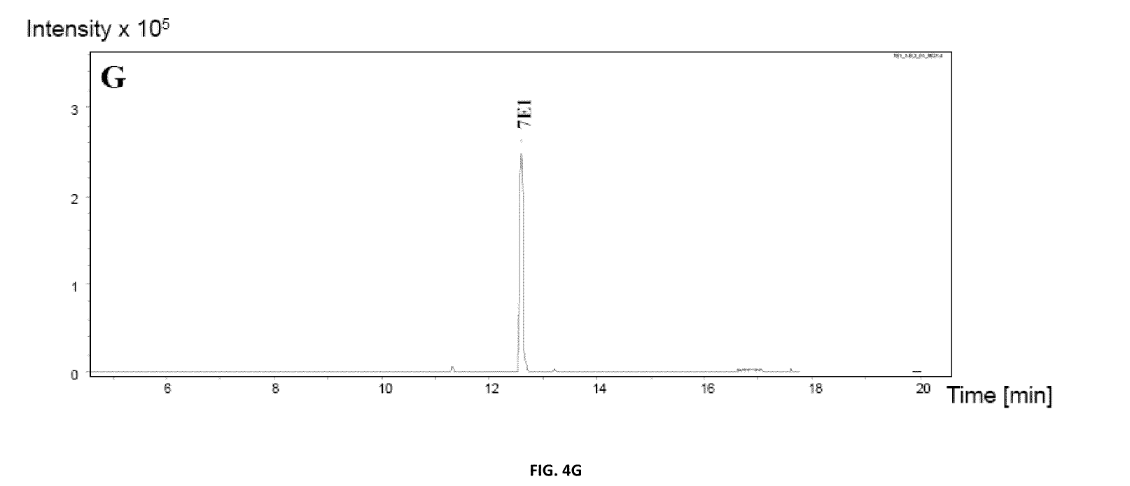

Six major purified compounds (compound 7B1, 7B5, 7C5, 7D7, 7E1 and 7E2) are isolated from Fraction 7 of gypenosides (900 mg) by ODS column (Gradient elution: 50-100%). These six compounds are further identified using chromatography over silica gel and MCI-gel CHP-20P respectively, which shows six main peaks of Fraction 7 as illustrated in FIG. 4 and Table 2.

TABLE 2
Compounds isolated from Fr. 7 of total gypenosides

| Compounds | Amount (mg) | tR (min) | [M − H]⁻ (m/z) |
|---|---|---|---|
| 7B5 | 27 | 9.7 | 961.5 |
| 7C5 (12) | 60 | 10.5 | 945.5 |
| 7B1 | 17 | 11.1 | 799.4 |
| 7D7 (14) | 150 | 11.6 | 931.5 |
| 7E2 | 40 | 12.2 | 915.5 |
| 7E1 (15) | 13 | 12.6 | 799.4 |

Different concentrations of these six compounds are further tested in the yeast cells with or without the presence of mevalonate (FIG. 5). As seen in the results shown in FIG. 5, compounds 7B1, 7B5, 7C5 and 7D7 have no to low inhibitory growth effect towards yeast cells. Moreover, no to low restoration of growth is observed in the presence of mevalonate in these four compounds. There are 65% and 80% growth inhibition in the yeast cells for compound 7E1 at concentration of 150 μg/ml and compound 7E2 at concentration of 300 μg/ml, respectively (FIG. 5). Importantly, significant reduction of inhibitory effects is observed in the presence of mevalonate (FIG. 5). Based on this yeast cell-based platform of the present invention, two potential HMG-CoAR inhibitors from GpS have been isolated.

Chemical Structure of Active Compounds Isolated from Fr. 7

Compounds 7E1 and 7E2 are active HMG-CoAR inhibitors identified and isolated according to the present invention. Compound 7E1 is a gypenoside XLV-Ed by comparing with the authentic compound and further confirmed by MS and NMR data. Compound 7E2 is a gypenoside IX based on MS and NMR data.

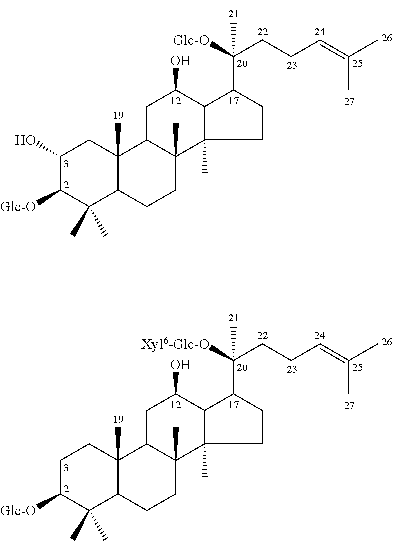

gypenoside XLV-Ed (7E1; Formula I) gypenoside IX (7E2; Formula II)

Compound 7E1: C₄₂H₇₂O₁₄, white powder. Negative HR-ESI-MS (m/z): 799.4870 [M-H]⁻ (calculated for C₄₂H₇₁O₁₄, 799.4844).¹H-NMR (pyridine-d5, 400MHz) : δ 0.90, 0.95, 0.98, 1.06, 1.41, 1.64 (each 3H, all s, H-18, 19, 21, 28, 29, 30), 1.59 (6H, s, H-26, 27), 3.28 (1H, d, J=9.3 Hz, H-3), 4.98 (1H, d, J=7.8 Hz, 3-O-glc-1), 5.21 (1H, d, J=7.8 Hz, 20-O-Glc-1), 5.26 (1H, brs, H-24), 5.58 (1H, s, H-12). ¹³C-NMR data are listed in Table 2. This compound was identified to be gypenoside XLV-Ed by comparing ¹H-NMR and ¹³C-NMR data with that recorded in Takemoto T, Arihara S, Yoshikawa K, Kawasaki J, Nakajima T, Okuhira M. Studies on the constituents of Cucurbitaceae plants XI. On the saponins constituents of Gynostemma pentaphyllum Makino (7). Yakugaku Zasshi, 1984, 104 (10): 1043-9.

Compound 7E2: C₄₉H₈₀O₁₇, white powder. Negative HR-ESI-MS (m/z): 915.5349 [M-H]⁻ (calculated for C₄₇H₇₉O₁₇, 915.5323). ¹H-NMR (pyridine-d5, 400MHz) : 0.81, 0.96, 0.98, 0.99, 1.32, 1.61, (3H each, all s, H-18, 19, 21, 28, 29, 30), 1.66 (6H, s, H-26, 27), 4.61 (1H, d, J=10.0 Hz, 3-O-Glc-6), 4.73 (1H, d, J=10.0 Hz, 20-O-Glc-6), 4.96 (1H, d, J=8.0 Hz, 3-O-Glc-1), 5.00 (1H, d, J=7.0 Hz, Xyl-1), 5.14 (1H, d, J=8.0 Hz, 20-O-Glc-1), 5.60 (1H, s, H-12). ¹³C-NMR data are listed in Table 3. This compound was characterized to be gypenoside IX by comparison of ¹H-NMR and ¹³C-NMR data with that described in Zuo G Y, Wei J X, Du Y C, Cao S M, Chen Y G. Studies on saponins from flower-buds of Sanchi (Panax notoginseng (Burk.) F. H. Chen). Natural Product Research and Development, 1991, 3 (4): 24-30.

TABLE 3
¹³C-NMR data (pyridine-d₅) of compounds 7E1 and 7E2

| C | 7E1 | 7E2 | | 7E1 | 7E2 |
|---|---|---|---|---|---|
| 1 | 48.4 | 39.7 | 3-O-Glc-1 | 107.1 | 107.4 |
| 2 | 67.4 | 27.3 | 2 | 76.2 | 75.4 |
| 3 | 95.9 | 89.3 | 3 | 79.3 | 79.2 |
| 4 | 41.4 | 40.2 | 4 | 72.2 | 72.4 |
| 5 | 56.7 | 56.9 | 5 | 79.2 | 78.5 |
| 6 | 19.0 | 19.0 | 6 | 63.2 | 63.6 |
| 7 | 35.6 | 35.6 | 20-O-Glc-1 | 98.8 | 98.6 |
| 8 | 40.6 | 40.5 | 2 | 75.7 | 75.3 |
| 9 | 50.8 | 50.7 | 3 | 80.0 | 79.8 |
| 10 | 38.4 | 37.4 | 4 | 72.2 | 72.0 |
| 11 | 31.6 | 31.3 | 5 | 78.8 | 76.3 |
| 12 | 70.6 | 70.7 | 6 | 63.5 | 70.5 |
| 13 | 50.1 | 50.0 | Xyl-1 | | 106.3 |
| 14 | 52.0 | 51.9 | 2 | | 74.6 |
| 15 | 31.3 | 31.2 | 3 | | 77.4 |
| 16 | 27.1 | 27.2 | 4 | | 71.6 |
| 17 | 52.1 | 52.1 | 5 | | 67.5 |
| 18 | 16.5 | 16.5 | | | |
| 19 | 18.0 | 16.8 | | | |
| 20 | 83.8 | 84.0 | | | |
| 21 | 22.8 | 22.8 | | | |
| 22 | 36.7 | 36.7 | | | |
| 23 | 23.7 | 23.7 | | | |
| 24 | 126.6 | 126.5 | | | |
| 25 | 131.5 | 131.6 | | | |
| 26 | 26.3 | 26.4 | | | |
| 27 | 18.5 | 18.5 | | | |
| 28 | 29.0 | 28.6 | | | |
| 29 | 18.3 | 17.3 | | | |
| 30 | 18.0 | 17.9 | | | |

### Example 5—In Vitro HMG-CoAR Activity Assay

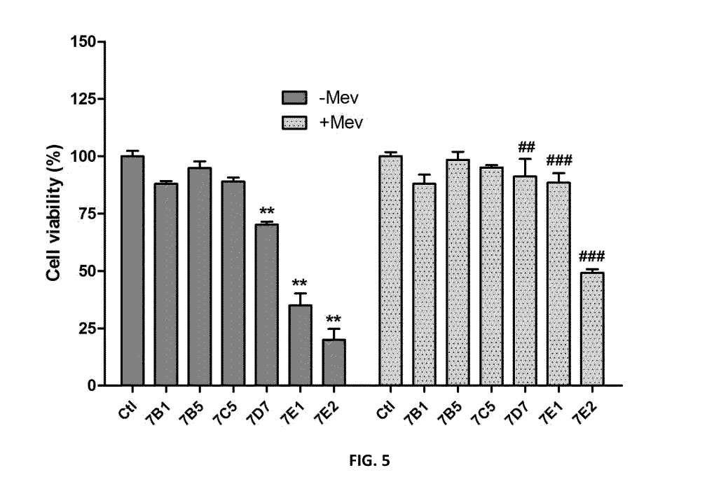

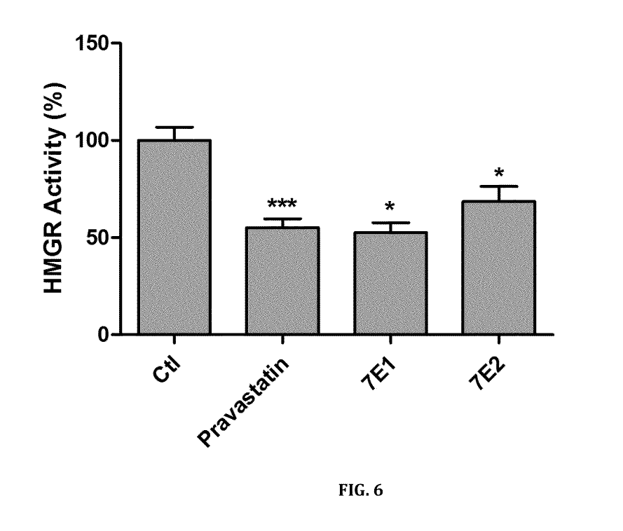

To validate the inhibitory effect of HMG-CoAR activity of compounds 7E1 and 7E2, in vitro HMG-CoAR activity is performed. Commercial HMG-CoAR activity assay kit (Sigma, USA) is used. The HMG-CoAR activity assay is based on the spectrophotometric measurement of the decrease in absorbance at 340 nm, which represents the oxidation of NADPH by the catalytic subunit of HMGR in the presence of the substrate of HMG-CoA. HMG-CoAR inhibitory effect of Pravastatin (Positive control), 7E1 or 7E2 are tested. Results show that compounds 7E1 and 7E2 can inhibit the HMG-CoAR activity (FIG. 6).

### Example 6—In Vivo effects of GpS in Reducing the Lipid and Total Cholesterol Levels

Elevation of Plasma Lipid Levels in Min Mice

C57BL/6J Min mice is used as a hyperlipidemia model. To confirm suitability of Min mice as a hyperlipidemia model, changes of plasma lipid levels, including triglyceride and total cholesterol levels, in the wild-type and Min mice of both genders at 16 weeks of age are determined and compared. Plasma lipids levels elevate significantly in Min mice compared with their wild-type counterparts (p<0.001). In female wild-type mice, the average value of triglyceride levels of plasma is 44.40±2.77 mg/dL, whereas in Min mice, the triglyceride levels is 513.27±114.75 mg/dL, 10.56 folds (p<0.001) higher than the wild type. The elevation is also found in the levels of total cholesterol, rising by 60.08% from 94.05±2.25 mg/dL to 150.56±11.09 mg/dL. The same observation is also found in male mice, the triglyceride levels increase 5.16-fold (p<0.001) in Min mice in contrast to that of the wild-type counterparts (from 57.42±5.54 mg/dL to 353.61±125.73mg/dL), and the total cholesterol levels of plasma in Min mice elevate 92.28% (p<0.001), from 106.01±0.92 mg/dL to 208.84±29.31 mg/dL. The observation of the elevated plasma lipid levels of Min mice indicates that the Min mice develop hyperlipidemia spontaneously, and therefore, it is an appropriate animal model to study the hyperlipidemia condition.

Reduction of Plasma Triglyceride Levels in Min Mice

In vivo effect of GpS to reduce lipid and total cholesterol level in treating hyperlipidemia or related condition is investigated using C57BL/6J Min Mice. Plasma triglyceride levels of Min mice having administrated 500 mg/kg GpS decrease compared to the control group without GpS administration. In females, the triglyceride levels reduce by 80.62% (p<0.05) from 513.27±114.73 mg/dL to 99.48±27.22 mg/dL. The same effect is also observed in male Min Mice, of which the triglyceride levels reduce by 67.93% (p<0.05) from 353.61±153.99 mg/dL to 113.39±21.47 mg/dL in the Min.

Reduction of Plasma Total Cholesterol Levels in Min Mice

Reduction of total cholesterol levels of plasma in Min mice is also observed after administration of 500 mg/kg GpS for four weeks in both genders. In female Min mice, the average total cholesterol levels was measured 150.56±11.09 mg/dL in the control group and reduced to 107.58±6.21 mg/dL after administration of GpS, and total cholesterol reduced in females was 28.55% (p<0.05). The same effect was observed in males, the state of hypercholesteremia was improved from 203.84±35.89 mg/dL to 122.17±7.29 mg/dL, decreasing by 40.07% (p <0.01).

The results indicate hyperlipidemic state is improved by administrating 500 mg/kg GpS, by which both triglyceride and total cholesterol levels in plasma reduce significantly after the four weeks treatment.

Based on the above Examples 1-6, the yeast-cell based activity-guided fractionation assay of the present invention provides a rapid, reproducible and low-cost assay to screen, identify and isolate novel HMG-CoAR inhibitors. The present invention is particularly useful in screening natural raw materials, such as herbs, as well as isolated chemical compounds. Further, the examples demonstrate that the total saponins from Gp or the isolated saponins from GpS have high levels of HMG-CoAR suppression and present novel treatment and/or prevention of both hyperlipidemic and hypercholesterolemia.

### INDUSTRIAL APPLICABILITY

The present invention discloses a method to identify inhibitors for the enzyme 3-hydroxy-3-methylglutaryl coenzyme A reductase (HMG-CoAR). In particular, the present invention relates to method for identifying and isolating HMG-CoAR inhibitors from natural products. This invention also provides novel HMG-CoAR inhibitors that are useful in the treatment and/or prevention of diseases and symptoms associated with increased cholesterol and/or blood lipid levels.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A method for screening to identify enzyme 3-hydroxy-3-methylglutaryl coenzyme A reductase (HMG-CoAR) inhibitors comprising:
providing a yeast strain which lacks HMG1 and HMG2 genes but containing a HMG1 overexpression plasmid;
contacting a test material to said yeast strain;
measuring cell viability of said yeast strain;
adding mevalonate, wherein the test material possesses HMG-CoAR inhibitory activity if the measured cell viability is reduced when the test material is introduced to the yeast strain without mevalonate but in the presence of mevalonate the cell viability is not reduced.

2. The method according to claim 1 wherein the method is provided in a yeast cell-based activity-guided fractionation assay screening.

3. The method according to claim 1 wherein the test material is a natural product.

4. The method according to claim 3 wherein the test material is a herb.

5. The method according to claim 1, further comprising separating the test material into at least two fractions and repeating said introducing, measuring and adding steps to identify the at least two fractions possess HMG-CoAR inhibitory activity.

6. The method of according to claim 5, further comprising isolating compounds from the at least two fractions which has been identified to possess HMG-CoAR inhibitory activity.

7. A kit for screening and identifying a material having HMG-CoR inhibitory activity according to the method of claim 1.

8. A kit for screening and identifying a material having HMG-CoAR inhibitory activity according to the method of claim 2.

9. A composition for treating a disease and/or condition associated with elevated cholesterol and/or blood lipid level comprising an effective amount of a extract of Gynostemma pentaphyllum (Gp), compounds isolated from Gp, total saponins from Gp, fraction from total saponins from Gp, isolated saponins from Gp, a compound having Formula (I) or (II) or a combination thereof, wherein Formula (I) and Formula (II) are

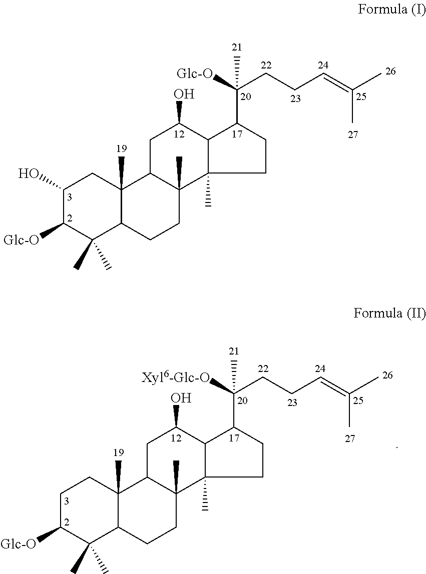

10. The composition of claim 9 wherein said composition is administered in conjunction with other compounds effective in treating diseases and/or condition associated with elevated cholesterol and/or blood lipid level.

11. The composition of claim 9, wherein the disease and/ or condition associated with elevated cholesterol and/or blood lipid level is hypercholesterolemia or hyperlipidemic.

12. A composition for preventing a disease and/ or condition associated with elevated cholesterol and/or blood lipid level comprising an effective amount of a extract of Gynostemma pentaphyllum (Gp), compounds isolated from Gp, total saponins from Gp, fraction from total saponins from Gp, isolated saponins from Gp , a compound having Formula (I) or (II), or a combination thereof, wherein Formula(I) and Formula (II) are

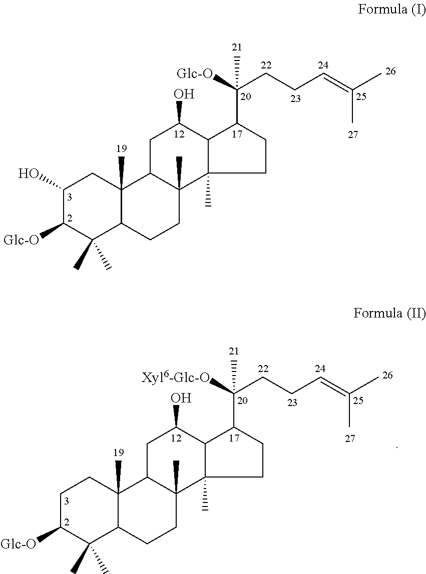

13. The composition of claim 12 wherein said composition is administered in conjunction with other compounds effective in preventing diseases and/or condition associated with elevated cholesterol and/or blood lipid level.

14. The composition of claim 12, wherein the disease and/or condition associated with elevated cholesterol and/or blood lipid level is hypercholesterolemia or hyperlipidemic.
